(19) United States
(12) Patent Application Publication
(10) Pub. No.: US 2015/0338485 A1
(43) Pub. Date: Nov. 26, 2015

(54) METAL PARTICLE MRI CONTRAST AGENTS AND METHODS OF USE

(71) Applicant: Georgetown University, Washington, DC (US)

(72) Inventors: Joseph N. YORK, Washington, DC (US); Edward van KEUREN, Herndon, VA (US); Christopher ALBANESE, Pelham Manor, NY (US); Olga RODRIGUEZ, Washington, DC (US); Yi-Chien LEE, Rockville, MD (US)

(73) Assignee: GEORGETOWN UNIVERSITY, Washington, DC (US)

(21) Appl. No.: 14/758,778

(22) PCT Filed: Dec. 30, 2013
(86) PCT No.: PCT/US2013/078236

§ 371 Date: Jun. 30, 2015

Related U.S. Application Data
(60) Provisional application No. 61/747,669, filed on Dec. 31, 2012.

(51) Int. Cl. G01R 33/56; C23C 14/34; C01G 49/00; A61B 5/055; A61K 49/18

(52) U.S. Cl. CPC G01R 33/5601; A61B 5/055; A61K 49/1866; C01G 49/0036; C23C 14/34; C01P 2006/42

(57) ABSTRACT

Disclosed herein is a novel MRI contrast agent. The MRI contrast agent is a metal particle having particular shape and size parameters that provides the metal particle with the ability to improve contrast by resisting transverse relaxivity saturation. The disclosed metal particle may be used in both medical and research diagnostic procedures, such as MRI, X-ray, and NMR. Also disclosed are compositions comprising the disclosed metal particle, and methods of using the metal particle. In particular disclosed embodiments, the metal particle is a barium ferrite particle.

### CROSS REFERENCE TO RELATED APPLICATION

This application claims the benefit of and priority to U.S. Provisional Application No. 61/747,669 filed on Dec. 31, 2012, which is incorporated herein by reference in its entirety.

### FIELD

The present disclosure concerns embodiments of a novel contrast agent for use in magnetic resonance imaging (MRI), methods of using the contrast agent, and methods of improving dispersion of the contrast agent in various media.

### BACKGROUND

Magnetic resonance imaging (MRI) is a non-invasive imaging technique with inherent soft-tissue contrast. Images are created from the relaxation times of the nuclear magnetic moments of the protons in water. These times can be either the longitudinal relaxation time, T₁, or the transverse (phase) relaxation time, T₂. The contrast in the image can be improved by the addition of materials that interact with the moments. For example, Gadolinium-based contrast agents are commonly used to improve resolution in images weighted by T₁ relaxation times. Ferromagnetic and superparamagnetic iron oxide nanoparticles (SPIONs) have recently been developed as T₂ contrast agents. The mechanism for the effect of the particles on the phase relaxation is the inhomogeneous magnetic field in the vicinity of the particle. It has been shown that the effect on T₂ times has a strong dependence on particle size for spherical particles. These findings indicate increasing particle size improves contrast up to a critical size, but further increasing the particle size is detrimental to the contrast agent effect on T₂.

### SUMMARY

Disclosed herein is a metal particle MRI contrast agent. In particular disclosed embodiments, the metal particle MRI contrast agent is a barium ferrite MRI contrast agent having a total hard axis ranging from about 30 nm to about 300 nm and a total easy axis of about 5 nm to about 50 nm. In particular disclosed embodiments, the barium ferrite may have a total hard axis ranging from about 30 nm to about 200 nm and a total easy axis of about 5 nm to about 50 nm. In other particular disclosed embodiments, the barium ferrite may have a total hard axis ranging from about 50 nm to about 150 nm and a total easy axis of about 10 nm to about 40 nm. In other disclosed embodiments, the barium ferrite may have a total hard axis ranging from about 75 nm to about 100 nm and a total easy axis of about 15 nm to about 30 nm. The barium ferrite may be an oblate disk, tube, or combination thereof. The disclosed oblate barium ferrite MRI contrast agent typically may produce an improved transverse relaxivity as compared with a sphere having a similar size. In particular disclosed embodiments, the transverse relaxivity may range from about 2000 mL/mg·s to about 2600 mL/mg·s. Even more typically, the transverse relaxivity may range from about 2100 mL/mg·s to about 2450 mL/mg·s. The barium ferrite MRI contrast agent may also comprise one or more pores.

Also disclosed herein is a pharmaceutical composition comprising the metal particle MRI contrast agent disclosed herein and a pharmaceutically acceptable agent. In particular disclosed embodiments, the metal particle MRI contrast agent is selected from barium ferrite. The pharmaceutically acceptable agent is selected from a component that promotes aqueous solubility, a component capable of binding to a target, a component that facilitates biological administration, and combinations thereof.

In particular disclosed embodiments, the component that promotes aqueous solubility is a polar ligand having a net negative or positive charge or a neutral polar ligand. The polar ligand typically may be selected from an optionally substituted ammonium species, an optionally substituted carboxylate species, an optionally substituted alkoxy species, an optionally substituted thio species, an optionally substituted phosphonates, an optionally substituted alkylene oxide, an optionally substituted thiol species, and combinations thereof. In particular disclosed embodiments, the polar ligand may be selected from mercaptoacetic acid, mercaptopropionic acid, dihydrolipoic acid, cetyl-trimethylammonium bromide, α-cyclodextrin, polyethylene glycol, cysteamine, cysteine, and the like.

In other disclosed embodiments, the component capable of binding to a target is a specific binding moiety. For example, the specific binding moiety may be selected from an antibody, a protein, a peptide, a nucleic acid sequence, an enzyme, or combinations thereof.

In other disclosed embodiments, the pharmaceutical composition may comprise a component that facilitates biological administration, such as a pharmaceutically acceptable excipient. Particular disclosed embodiments include surfactants, carbohydrates, lubricants, buffers, osmolality agents, and combinations thereof.

In certain embodiments of the disclosed composition, the pharmaceutically acceptable agent may be coupled to the barium ferrite particle. The pharmaceutically acceptable agent may be coupled electrostatically, covalently, or combinations thereof.

Further embodiments concern a method for increasing T₂ contrast in magnetic resonance imaging, comprising providing the metal particle MRI contrast agent disclosed herein, and exposing the metal particle MRI contrast agent to a magnetic field. In other embodiments, the T₂ contrast may be increased by providing the composition disclosed herein and exposing the composition to a magnetic field.

Yet other embodiments concern an improved MRI contrast agent comprising a barium ferrite nanoparticle, the improvement being the barium ferrite nanoparticle having an oblate shape with a magnetic axis that is perpendicular to a magnetic field, the oblate shape providing the barium ferrite nanoparticle with the ability to influence T₂ relaxation without saturation.

Another embodiment disclosed herein concerns a method for improving dispersion of the metal particle MRI contrast agent disclosed herein, comprising modifying the metal particle MRI contrast agent's magnetic properties, modifying the metal particle MRI contrast agent's ability to aggregate, or combinations thereof. The metal particle MRI contrast agent's ability to aggregate may be modified by encapsulating the metal particle within a polymer colloid, attaching one or more ligands to the metal particle, exposing the metal particle to ultra-sonication and/or high-shear colloid milling, and combinations thereof. In other embodiments, the metal particle MRI contrast agent's magnetic properties may be modified by changing the metal particle from a ferromagnetic particle to a superparamagnetic particle by flipping magnetization using temperature control.

Yet another disclosed embodiment concerns an improved nanoparticle suspension for use in MRI diagnostic procedures, the improvement comprising oblate barium ferrite nanoparticles suspended in a medium that prevents nanoparticle aggregation thereby allowing for more accurate imaging by producing varying field gradients capable of magnetically influencing surrounding water molecules and thereby producing an increased signal.

Also disclosed herein is a method, comprising administering to a subject an effective amount of the metal particle MRI contrast agent disclosed herein, exposing the subject, or a tissue or cell of the subject, to a magnetic field, and detecting a signal produced by the interaction between the metal particle MRI contrast agent, the magnetic field, and a target within the subject, the tissue, or cell. The metal particle MRI contrast agent may be administered intravenously or orally. In particular disclosed embodiments, the effective amount of the metal particle MRI contrast agent ranges from about 0.01 mmol/kg to about 0.1 mmol/kg, even more typically from about 0.01 mmol/kg to about 0.075 mmol/kg, and even more typically from about 0.01 mmol/kg to about 0.05 mmol/kg. Detecting the signal typically concerns obtaining a magnetic resonance image of the subject and/or the particular target. The image may be a T₁ weighted image, or it may be a T₂ weighted image.

The foregoing and other objects, features, and advantages of the present disclosure will become more apparent from the following detailed description, which proceeds with reference to the accompanying figures.

### BRIEF DESCRIPTION OF THE DRAWINGS

FIG. 1 is an image illustrating various shapes and dimensions of particular disclosed embodiments of the particle disclosed herein.

FIG. 2 is a graph of transverse relaxation rates (1/s) as a function of particle concentration (mg particle/mL gel).

FIG. 3 is a graph of transverse relaxivity (mL/mg·s) as a function of particle surface area-to volume-ratio (1/m).

FIG. 4 is a SEM image of a particular embodiment of a spherical particle having a mean radius of about 6 nm.

FIG. 5 is a SEM image of a particular embodiment of a spherical particle having a mean radius of about 16 nm.

FIG. 6 is a SEM image of a particular embodiment of a prolate-shaped particle having half axes lengths of about 7 nm (magnetic hard axis) and about 24 nm (magnetic easy axis).

FIG. 7 is a SEM image of a particular embodiment of a prolate-shaped particle having half axes lengths of about 15 nm (magnetic hard axis) and about 225 nm (magnetic easy axis).

FIG. 8 is a SEM image of a particular embodiment of an oblate-shaped particle having half axes lengths of about 100 nm (magnetic hard axis) and about 10 nm (magnetic easy axis).

FIG. 9 is an image of a contrast agent sample comprising multiple layers of agar containing varying concentrations of the disclosed particle.

FIG. 10 is an image illustrating a particular slice of the contrast agent sample through which imaging analysis is performed. The image illustrates three different views of the sliced portion: a front view (a); a side view (b), and a top view (c).

FIG. 11 is a T₂-weighted image of various different contrast agent samples. Each square within the image illustrates the area used to calculate T₂ in each layer.

FIG. 12 is a graph of transverse relaxation rates (1/s) versus particle concentration (mg/g). This particular graph compares results obtained from particular disclosed working embodiments and results previously obtained in the art.

### DETAILED DESCRIPTION

### I. Introduction

The following term definitions are provided to aid the reader, and should not be considered to provide a definition different from that known by a person of ordinary skill in the art. And, unless otherwise noted, technical terms are used according to conventional usage.

As used herein, the singular terms “a,” “an,” and “the” include plural referents unless context clearly indicates otherwise. Similarly, the word “or” is intended to include “and” unless the context clearly indicates otherwise. Also, as used herein, the term “comprises” means “includes.” Hence “comprising A or B” means including A, B, or A and B.

All publications, patent applications, patents, and other references mentioned herein are incorporated by reference in their entirety for all purposes. In case of conflict, the present specification, including explanations of terms, will control.

Although methods and materials similar or equivalent to those described herein can be used to practice or test the disclosed technology, suitable methods and materials are described below. The materials, methods, and examples are illustrative only and not intended to be limiting.

In order to facilitate review of the various embodiments of the disclosure, the following explanations of specific terms are provided:

Ferromagnetic: A form of magnetism whereby certain materials form permanent magnets or are attracted to magnets.

Oblate: A spheroid, such as a rotationally symmetric ellipsoid, that has a polar axis shorter than the diameter of the equatorial circle whose plane bisects the polar axis.

Paramagnetism: A form of magnetism whereby materials are only attracted when in the presence of an externally applied magnetic field. The materials do not retain magnetic properties when the external magnetic field is removed.

Phantom: A sample containing a homogeneous medium to test the performance of an MRI machine, or the effects of a contrast agent on a homogeneous medium MR image. A phantom may be of any form, and typically is an agar.

Prolate: A spheroid in which the polar axis is larger than the diameter of the equatorial circle whose plane bisects the polar axis.

Metal Particle: As used herein, this term refers to a contrast agent that comprises a metal species and may refer to any metal-containing species suitable for diagnostic methods selected from MRI, NMR, X-ray, and the like. In particular disclosed embodiments, the metal particle is a metal oxide and/or a metal ferrite.

MRI: Magnetic resonance imaging.

MRI contrast agent: An agent that increases the contrast between different portions of a sample by altering relaxation times, T₁ and T₂, during magnetic resonance imaging. MRI contrast agents are classified by the different changes in relaxation times after they have been added to a sample.

Spher(e/ical): A substantially round geometrical object in three-dimensional space wherein a set of points are all the same distance r from a given point in space.

Spheroid: A quadric surface obtained by rotating an ellipse about one of its principal axes.

Subject: Refers to humans and non-human subjects, such as mammals.

T₁ relaxation: Known as the longitudinal (or spin-lattice) relaxation time. T₁ is the decay constant for the recovery of the z component of the nuclear spin magnetization, M_{z}, towards its thermal equilibrium value, M_{z,eq}.

T₂ relaxation: Known as the transverse (or spin-spin) relaxation time. T₂ is the decay constant for the component of the nuclear spin magnetization vector, M, perpendicular to the external magnetic field, B₀.

### II. Particles

The present disclosure concerns embodiments of a metal particle that may be used as a novel T₁ or T₂ contrast agent in MRI applications. Disclosed embodiments of the particle have unique shape and size characteristics that together allow the metal particle to act as a suitable T₁ or T₂ contrast agent. The disclosed metal particle typically comprises one or more metals and a suitable counter ion. In particular disclosed embodiments, the metal particle is a metal oxide or a metal ferrite. The metal may be selected from iron, barium, cobalt, strontium, zirconium, zinc, and combinations thereof. Typical particles include, but are not limited to barium ferrite, cobalt ferrite, strontium ferrite, zirconium ferrite, zinc ferrite, iron oxide, and barium oxide.

Particular disclosed embodiments concern a barium ferrite nanoparticle that acts as an effective and improved contrast agent for MRI analysis. Barium ferrite is known in the art as a common magnetic recording medium; however, the particular barium ferrite particles disclosed herein illustrate the ability to act as effective contrast agents in MRI applications. In particular disclosed embodiments, this ability is caused by the ability of the size and shape of the barium ferrite nanoparticle to effect relaxivity of its surrounding environment (e.g., protons in surrounding water molecules).

The disclosed metal particle may have a particular shape and size that allows the particle to change the spatial distribution of the magnetic field surrounding the particle. In particular disclosed embodiments, the particle has a unique field that is attributed to its particular shape. This unique field may influence the homogeneity of the magnetic field applied in a particular apparatus, such as the magnetic field applied in an MRI apparatus. By influencing the homogeneity of the applied magnetic field, the particle may alter the T₂ relaxation behavior of surrounding protons, such as water protons. In particular disclosed embodiments, the shape of the magnetic particle may influence the particle's effect on its surrounding environment. In other disclosed embodiments, the size of the magnetic particle may influence the particle's effect on its surrounding environment. In yet other disclosed embodiments, the combination of the size and the shape of the magnetic particle substantially increases the particle's ability to influence its surrounding environment. It is currently believed that the combination of the shape and size of the particle contributes to the particle's ability to act as a contrast agent.

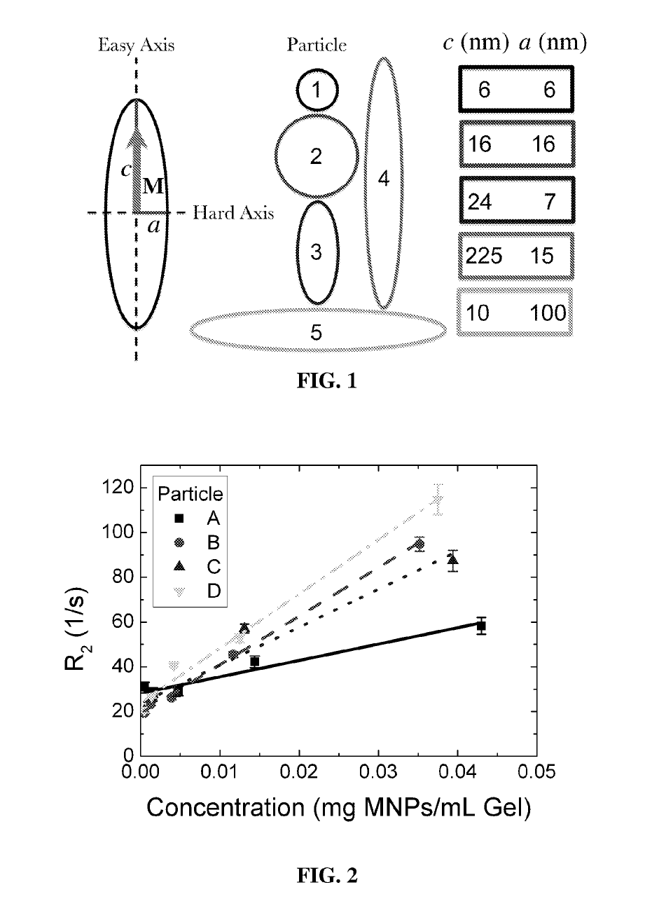

The shape of the disclosed particle may be substantially spherical, spheroidal, flat, square, rectangular, conical, hexagonal, and needle-like. In particular disclosed embodiments, the metal particle may be oblate, prolate, or a flat disc. Exemplary embodiments may have the shapes and dimensions provided in FIG. 1

The size of the disclosed particle may be varied in order to obtain different relaxivity values. For example, in particular disclosed embodiments, the particle may have a total hard axis ranging from about 30 nm to about 300 nm and a total easy axis of about 5 nm to about 50 nm. In particular disclosed embodiments, the barium ferrite may have a total hard axis ranging from about 30 nm to about 200 nm and a total easy axis of about 5 nm to about 50 nm. More typically, the particle has a total hard axis ranging from about 50 nm to about 150 nm and a total easy axis of about 10 nm to about 40 nm. In other disclosed embodiments, the particle may have a total hard axis ranging from about 75 nm to about 100 nm and a total easy axis of about 15 nm to about 30 nm.

The effect of particle shape and/or size may be determined by performing magnetic imaging analysis (e.g., MRI analysis), obtaining relaxation times for one or more samples, plotting the inverse of the relaxation time versus concentration of the sample, and then determining the relaxivity for each sample. In particular disclosed embodiments, plots of the relaxivities as a function of particle shape or size independently may or may not illustrate a relationship. In particular disclosed embodiments, no relationship between shape and relaxivity or size and relaxivity is observed. However, when both properties are examined simultaneously, a noticeable relationship between relaxivity and particle shape and size may be observed. Exemplary results are provided in FIG. 2.

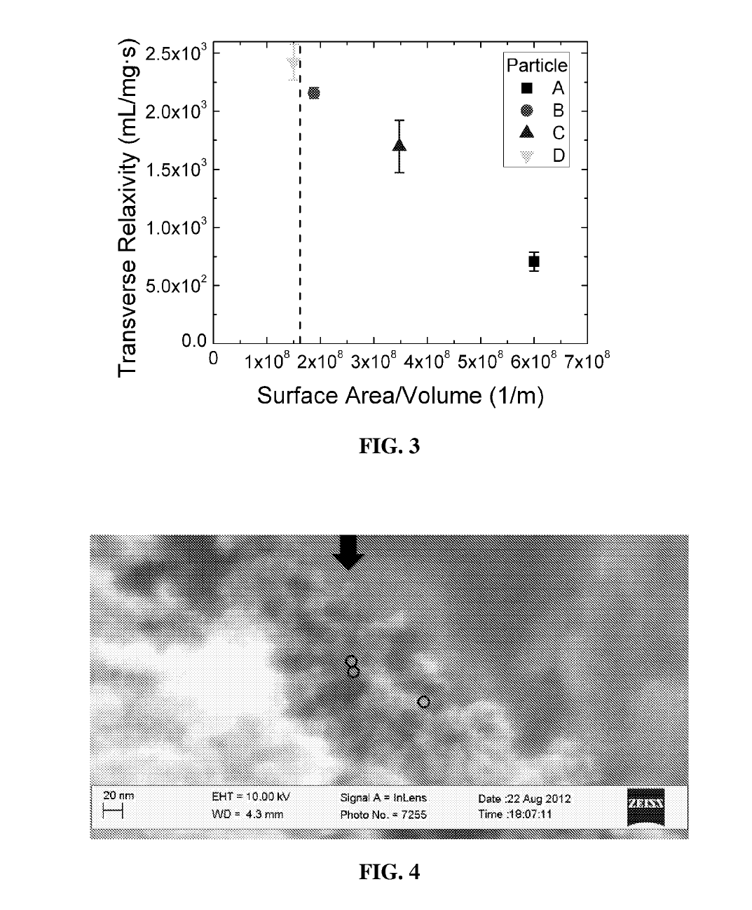

The effects of size and shape of the particle on relaxivity also may be determined by analyzing the surface area-to-volume ratio. A relaxivity (e.g., 1/R) dependence has been reported in the art for spherical contrast agents; however, the present disclosure illustrates that this dependence appears to apply to spheroids in general and not just spheres, if the 1/R is generalized to surface area-to-volume. For example, FIG. 3 illustrates a linear relationship between the relaxivities and surface area to volume ratio. In particular disclosed embodiments, an oblate disc-shape (e.g., particle D) caused the greatest effect. Such an effect is surprising and unexpected given the fact that a sphere of similar size is too large to cause such a strong response; a characteristic that is understood in the art. The magnitude of the response obtained from this particular disclosed embodiment is comparable to that of an ideal sphere, as indicated in FIG. 3 (vertical dashed line). Without being bound to a particular theory, it is currently believed that the shape of the particle allows more water molecules in the vicinity of the particle to experience the field inhomogeneities, as discussed previously herein. Accordingly, size and shape both affect the transverse relaxivity and the particular shape and size of particular particles disclosed herein allows the particle to act an effective contrast agent.

In particular disclosed embodiments, particle shape and crystal structure both affect the particle magnetization orientation. The magnetization induces an internal field, called the demagnetization field. This field is shape dependent and the energy is minimized when the magnetization lies along the axis of this field (the easy axis), which is along the particle major axis (the longer axis).

The crystal structure of the particle also affects the magnetization. Energy is also minimized when the magnetization lies along the crystal easy axis. Both shape and crystal effects determine the direction of magnetization. In particular disclosed embodiments (e.g., Particles 3 and 4), shape effects cause the magnetization to lie along the particle major axis. In other disclose embodiments, (e.g., Particle 5), the particle may have a strong crystal easy axis, which causes the magnetization lie normal to the disc plane. In particular disclosed embodiments, the particle material was changed in order to have a particle whose magnetization lies perpendicular to the shape easy axis.

Another feature of the disclosed particle is that it may comprise one or more pores suitable for housing one or more water molecules. In particular disclosed embodiments, the presence of the pore within the particle allows it to have a wider range of field strength and thereby increase the inhomogeneous magnetic field in the vicinity of the particle.

The relaxivities from various embodiments of the particle may be obtained and then plotted as a function of particle surface area (a particle parameter dependent upon shape) divided by volume (a particle parameter dependent upon size). A strong linear relationship may be observed between these two particle parameters and transverse relaxivity. In particular disclosed embodiments, the transverse relaxivity may increase as the ratio of surface area-to-volume decreases. Results from exemplary embodiments are illustrated in FIG. 3. As illustrated in FIG. 3, the linear relationship between the size and shape of the particle and the transverse relaxivity is obtained.

Theoretical work known in the art concerning spherical particle contrast agents indicates that increasing the size of a spherical particle contrast agent above a certain radius should decrease the relaxation rate of the medium about the particle when using a CPMG spin echo imaging technique. In particular, the effects of diffusion about the particle should be reduced for larger particles. The water protons in the surrounding medium would not traverse large enough lengths to dephase due to the particle field gradient; an undesirable effect for MRI applications. The maximum relaxation rate for particular disclosed particles (e.g., magnetite) occurs at the critical radius R_{c}=19 nm, according to work currently existing in the field. The surface area-to-volume ratio for a sphere with radius R_{c} is 1.58·10⁸ l/m. In particular disclosed embodiments, this hypothetical ratio was found to exist between the ratios obtained for particular disclosed embodiments of the particle disclosed herein. In particular, the hypothetical ratio was found to be between those obtained for Particles D and B, as illustrated in FIG. 3 (the dashed line represents the theoretical ratio). Accordingly, particular disclosed embodiments of the particle can produce important relaxivity responses in comparison to a spherical particle having a maximum radius.

Particular disclosed embodiments of the magnetic particle discussed herein may have a surface area-to-volume ratio that is smaller that of a sphere with radius R. Such embodiments also may have a much larger volume. However, rather than decrease the effect on the surrounding environment, these particular particles may have a stronger influence on the relaxation rate than spheres with a mean radius R=16 nm, which is close to the critical radius for particular disclosed embodiments, such as magnetite (e.g., particle B). The current spherical model used in the art does not provide an explanation for why a larger oblate spheroid has a significant effect on the transverse relaxation rate of agar where spheres of this size do not produce such large effects. The present disclosure illustrates that there is an association between particle shape and size affecting transverse relaxivity. The strong linear relationship in the data of FIG. 3 shows this dependence.

In particular disclosed embodiments, an oblate barium ferrite platelet was investigated, which had a surface area-to-volume ratio equivalent to a sphere with radius R=20 nm, but a volume 12.5 times larger than a corresponding sphere. In other disclosed embodiments, a prolate magnetite spheroid was investigated, which had a surface area-to-volume ratio equivalent to a sphere with radius R=8.62 nm, but a volume approximately 1.84 times larger than the corresponding sphere. In particular disclosed embodiments, the prolate and/or oblate spheroid samples may exhibit similar results obtained from the spherical model known in the art, which supports the hypothesis that shape is an important parameter to consider for contrast agents and that spheroids are potential candidates for MRI applications. In particular, the results disclosed herein indicate that large, non-spherical particles can be formed and are able to produce strong relaxation effects that spherical particles of equivalent size cannot produce using spin echo imaging sequences.

The current state of the art indicates that the general trend for spherical contrast agents is that given the same amount (or mass) of magnetic material, a smaller quantity of larger particles provides a greater effect on contrast than an abundance of smaller particles. This observation is attributed to fewer larger particles being more dispersed within the containing medium so that their field gradients do not overlap. This dispersion decreases the probability that water protons can diffuse between particles, and thus move back into a region of similar field strength which negates the diffusional dephasing. Given this observation, a desirable contrast agent should have fewer larger particles with field gradients that envelope a larger volume provided that the field strength still reduces appreciably over small distances about the particle so that the water protons appreciably dephase due to diffusion. Non-spherical particles have field gradients that extend further, as compared with spherical particles, in the directions parallel to the particle magnetization and are more closely condensed about the particle in the perpendicular directions compared to a spherical particle of similar volume. This change in field gradient causes a desirable effect on water protons diffusing near particular particles disclosed herein, particularly for the purposes of MRI using CPMG spin echo techniques.

### III. Compositions

The particle disclosed herein may be combined with one or more other pharmaceutically acceptable agents useful for medical diagnostic procedures. In particular disclosed embodiments, the particle is combined with other components that help promote aqueous solubility. In other disclosed embodiments, the particle may be combined with a component capable of binding to a target, e.g., a specific binding moiety. In yet further embodiments, the particle may be combined with a component that facilitates biological administration, such as a pharmaceutically acceptable excipient. The particle may be coupled with one or more of these disclosed components. In particular disclosed embodiments, the particle may be covalently coupled with the component. In other disclosed embodiments, the particle may be electrostatically coupled with the other component.

Non-limiting examples of a component that may be coupled with the particle in order to help promote aqueous solubility include polar ligands, such as a ligand comprising a net negative or net positive charge. Particular disclosed embodiments concern using optionally substituted ammonium species, optionally substituted carboxylate species, optionally substituted alkoxy species, optionally substituted thio species, optionally substituted phosphonates, and the like. The polar ligand also may be a polar neutral species, such as an optionally substituted alkylene oxide, optionally substituted thiol species, and the like. Exemplary ligands include mercaptoacetic acid, mercaptopropionic acid, dihydrolipoic acid, cetyl-trimethylammonium bromide, α-cyclodextrin, polyethylene glycol, cysteamine, cysteine, and the like.

Non-limiting examples of a component that may be coupled with the particle in order to specifically direct the particle to a particular target include specific binding moieties, such as antibodies, proteins, peptides, enzymes, and/or nucleic acid sequences.

Additionally, non-limiting examples of a component that facilitate biological administration include pharmaceutically acceptable excipients, which typically are substances suitable for biological administration. Non-limiting examples of pharmaceutically acceptable excipients include surfactants (e.g., dioctyl sodium sulfosuccinate (AOT), sodium dodecyl sulfate (SDS), Tween, Tergitol, Triton); carbohydrates (e.g., monosaccharides, disaccharides, polysaccharides, and the like); lubricants (e.g., talc, silica, fats, and the like), buffers (e.g., Tris, HEPES, TES, MOPS, SSC, TAPS, Bicine, Tricine, TAPSO, PIPES, succinic acid, and the like), osmolality agents (e.g., sodium chloride, propylene glycol, and the like), and combinations thereof.

### IV. Method of Making the Particle

Disclosed herein is a method of making the disclosed particle. In particular disclosed embodiments, the particle may be made using methods known to those having ordinary skill in the art, such as coprecipitation, sonochemical methods, reduction, microemulsion techniques, hydrothermal synthesis, and the like.

An example of a synthesis technique is provided herein. The particle may be produced by placing a metal halide species into a basic solution (e.g., aq. sodium hydroxide) and then adding an acidic solution (e.g., aq. HCl) to the metal-containing solution in a drop wise fashion. The solution is stirred and heated, and then the solution is exposed to a magnet and cooled. The particles are then isolated from the supernatant and washed with an acidic solution (e.g., HNO₃). The particles are then exposed to a similar magnetization/cooling process and the washing steps repeated. The particles are then dried.

Another example of particle synthesis concerns sonochemical preparation. In particular disclosed embodiments, a metal acetate species and a cyclic sugar species (e.g., cyclodextrin) are added to deoxygenated water. The solution is then sonicated with a high-intensity probe during which the water is continuously deoxygenated. The solution is then exposed to a magnet and allowed to cool. The particle is washed using deoxygenated water, and the washing is repeated until the supernatant is clear. The particles are then dried.

Yet another example of particle synthesis concerns reducing a starting material (e.g., goethite particles) to the desired particle using heat. In particular disclosed embodiments, a starting material is produced by mixing a solution of a metal sulfate with a basic solution (e.g., sodium hydroxide), and heating and stirring the resulting mixture. The precipitate is removed from the solution, washed with water, and then heated at a temperature of approximately 350° C. for a suitable time period (e.g., 3 hours). The resulting black powder is then cooled.

The particle may be further functionalized by coupling the particle with one or more of the other components disclosed herein. For example, the components may be coupled using ligand exchange methods, coupling reactions, and the like. The type of method used for coupling the component to the particle typically depends on the particular component being used.

In particular disclosed embodiments, the particle may be further manipulated in order to improve the particle's dispersion in solution or other medium. Disclosed embodiments of the particle are ferromagnetic and therefore may aggregate. It is desirable to have a particle that does not aggregate in MRI applications. Accordingly, the dispersion of the disclosed particle may be improved by various methods that either affect the magnetization of the particle and/or influence its interactions with its surrounding environment. For example, the particle may be converted from a ferromagnetic particle into a super paramagnetic particle by modifying the orientation of magnetization, such as through modifying the anisotropy of the particle, influencing the particle with temperature variation, and/or DC diode sputtering. In other disclosed embodiments, the particle's interactions with the surrounding environment may be influenced, such as by encapsulating the particle in a polymer colloid, attaching one or more of the ligands disclosed herein to the metal particle, exposing the metal particle to ultra-sonication and/or high-shear colloid milling, and combinations thereof.

### V. Method of Using the Particle

In particular disclosed embodiments, the particle is used as a contrast agent for medical diagnostic methods; however, the present disclosure is not limited to the medical field and may also be used for research purposes. Particular disclosed embodiments of the metal particle may be used in techniques selected from, but not limited to, MRI, NMR, X-ray, and the like. These diagnostic methods may be performed for medical diagnosis and/or for research purposes. In particular disclosed embodiments, the particle provides an improved signal as compared with other particles typically used in the art. In particular, the particle's shape and size both contribute to the particle's improved properties as a contrast agent as the shape and size of the particle allows for signal generation without saturation of relaxivity.

The particle may be used as a composition comprising any of the components disclosed herein. In particular disclosed embodiments, the particle may be formulated for administration to a subject. For example, the particle may be formulated as a solution suitable for intravenous administration, oral administration, or combinations thereof. The particle solution may be administered all at once or over a period of time. The subject typically is human; however, other types of subjects (e.g., domestic animals, veterinary animals, research animals, and the like) are contemplated.

Any tissue within the subject that is compatible with MRI imaging may be analyzed using the disclosed particle. In particular disclosed embodiments, the particle is effective in imaging tissues selected from organs, glands, nodes, connective tissue, muscle tissue, nervous tissue, epithelial tissue. In other disclosed embodiments, the particle is suitable for imaging bone. Additionally, the particle is useful for imaging tumors and other growths, including malignant and benign tumors.

After administration to the subject, imaging of the contrast agent may be performed, such as MRI imaging. In particular disclosed embodiments, a suitable period of time is allowed to pass between administration and imaging in order to allow the particles to disperse and/or migrate to particular areas of the body. In particular disclosed embodiments, the location of the particle in the subject may be controlled by combining the particle with one or more specific binding moieties capable of recognizing a target. Suitable specific binding moieties are disclosed herein.

The imaging may be carried out in vivo, in vitro, and/or ex vivo. In particular disclosed embodiments, the imaging may be performed on a sample that has been excised from a subject via surgery, biopsy, retraction, and other similar procedures. In other disclosed embodiments, imaging may be performed on cells and/or tissue in an in vitro setting, such as where the tissue and/or cell has been cultured.

When a magnetic field is applied during the imaging process, a detectable signal is produced. This detectable signal arises from interactions between the disclosed particle, the surrounding environment, and the applied magnetic field. In particular disclosed embodiments, the disclosed particle disrupts relaxation rates of water protons that are excited by the magnetic field. This disruption changes the contrast of the image obtained and thereby provides the ability to locate and/or visualize particular targets.

The amount of the particle that may be administered in the disclosed method typically is any amount effective to produce a detectable signal during MRI imaging analysis. In particular disclosed embodiments, the effective amount ranges from about 0.01 mmol/kg to about 0.1 mmol/kg; more typically from about 0.01 mmol/kg to about 0.075 mmol/kg; even more typically from about 0.01 mmol/kg to about 0.05 mmol/kg.

During imaging, a signal produced by the interaction between the particle, the applied magnetic field, and the surrounding environment may be detected. In particular disclosed embodiments, detecting the image comprises obtaining a magnetic resonance image of the subject. The magnetic resonance image may be a T₁ weighted image or a T₂ weighted image.

### VI. Working Embodiments

Five different types of magnetic particles were investigated. The particles are modeled as spheroids. Their shapes and dimensions (confirmed by SEM) are given in FIG. 1. The shapes are drawn such that the magnetization points up, and rotating the schematic about the particle magnetic easy axis (the vertical axis) gives the overall shape. With reference to FIG. 1, “c” is one half of the magnetic easy axis length, and “a” is one half of the magnetic hard axis length of the particle. Particles 1 through 4 are made of magnetite (Fe₃O₄), a ferromagnetic substance with a cubic crystal structure. Particle 5 is made of barium ferrite (BaFe₁₂O₁₉), a ferromagnetic substance with a hexagonal crystal structure.

### Particle Synthesis

Particles 2 and 5 were purchased from a commercial supplier. Particles 1, 3, and 4 were synthesized using already established techniques known in the art.

### Example 1

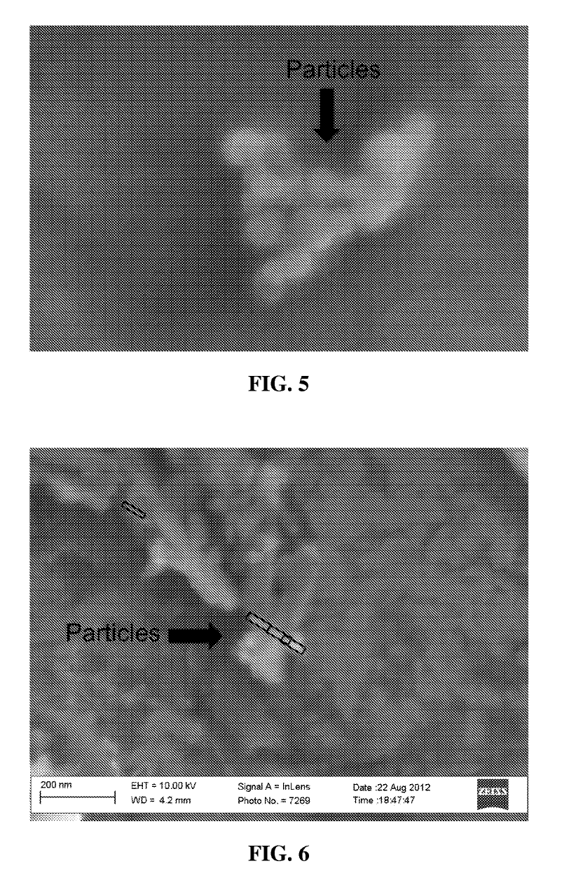

Particle 1 was prepared with a co-precipitation technique similar to those known in the art by placing a 2:1 molar ratio of ferrous and ferric chloride in a sodium hydroxide solution. For this 400 mL of 0.75M NaOH, made from sodium hydroxide crystals (Fisher Scientific) and distilled water, were placed in a 2 L beaker and heated to 100° C. while being continuously agitated with a stirring rod rotating at 550 rpm. Next, 40 mL of 1M FeCl₃ and 10 mL of 2M FeCl₂ were added to the solution. These solutions were prepared using FeCl₃.6H2O and FeCl₂.4H₂O (both Fisher Scientific) in distilled water, respectively. Following this, 20 mL of 2M HCl (Sigma Aldrich) was added drop wise. The contents were agitated and heated for ten minutes. After this time the beaker was placed on a permanent magnet and the contents of the beaker were allowed to cool to room temperature. The supernatant was then siphoned off and 200 mL of 1M HNO₃ (Acros Organics) was used to wash the particles. The mixture of particles and HNO₃ was agitated using a stirring rod rotating at 700 rpm for ten minutes. Afterwards the beaker was placed on the permanent magnet, the contents allowed to settle, and then the supernatant was removed. This washing process was repeated five times. After this process the particles were dried and placed in storage vials. An SEM image of Particle 1 is provided in FIG. 4. Additionally, an SEM image of Particle 2, another spherical magnetite, is provided in FIG. 5.

### Example 2

Particle 3 was prepared using a sonochemical method known in the art. Initially, 100 mL of distilled water was placed in a 250 mL beaker and deoxygenated by bubbling nitrogen gas through the water for ten minutes. Afterwards, 1 g of iron(II) acetate (Strem Chemicals) and 20 mg of β-cyclodextrin (Acros Organics) were added to the beaker. The contents were sonicated with a high-intensity probe (Fisher Scientific Model 550 Sonic Dismembrator, horn model CL4), set to power level 6, at room temperature and atmosphere pressure for 3 hours. Nitrogen gas was bubbled through the mixture throughout the sonication process. After sonication, the beaker was placed on a permanent magnet and the contents were allowed to cool to room temperature. Once cooled, the supernatant was removed and the particles were washed repeatedly by swirling them in 100 mL of distilled/deoxygenated water for five minutes and then setting the beaker on a permanent magnet. Washing was repeated until the supernatant was clear. After this the particles were dried and placed in storage vials. An SEM image of Particle 3 is provided in FIG. 6.

### Example 3

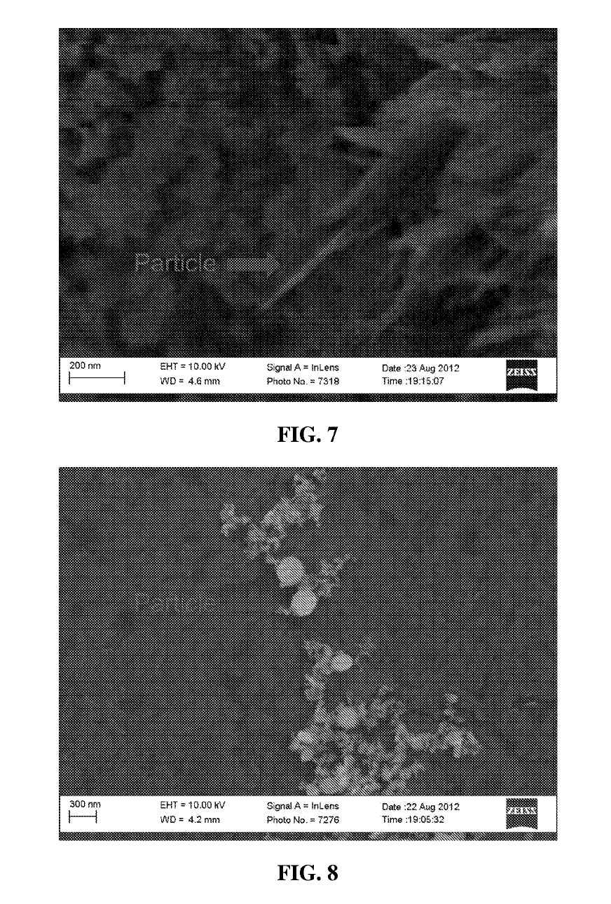

Particle 4 was prepared by creating acicular goethite particles and reducing them to magnetite by baking them in a furnace. The goethite particles were prepared in the following manner based on Matsui's method. In a 2 L beaker 62.6 g of FeSO₄.7H₂O (MP Biomedicals) was added to 250 mL of distilled water to create a 0.45 M FeSO₄ solution. This solution was agitated with a magnetic stirring rod rotating at 500 rpm. In a 500 mL beaker 43.3 g of NaOH crystals (Fisher Scientific) were placed in 225 mL of distilled water and dissolved by agitating with a magnetic stirring rod at 500 rpm. Once the sodium hydroxide solution was made, it was placed in the 2 L beaker with the ferrous sulfate solution. Distilled water was added to the beaker until a total volume of 500 mL was achieved. The 2 L beaker was then heated to 45° C. and agitated at 500 rpm. This temperature and agitation were maintained for four hours. Afterwards, the contents of the beaker were allowed to settle overnight. The yellow precipitate was then removed from the clear supernatant and washed using distilled water. The water was siphoned off, and the beaker was placed in an oven at 100° C. to dry. Once the particles were dry, the furnace temperature was increased to 350° C. and the precipitate was baked for three hours at this temperature. The beaker was shaken every half an hour to agitate the powder. After baking, the beaker and now black precipitate were allowed to cool to room temperature. The black powder was then placed in vials and labeled. An SEM image of Particle 4 is provided in FIG. 7. Additionally, an SEM image for Particle 5 is provided in FIG. 8. Exemplary particles and their measurements are provided in Table 1.

TABLE 1
Magnetic particles

| | | | | Contrast | Power |
|---|---|---|---|---|---|
| | | c | a | Agent | Absorption |
| Particle | Composition | (nm) | (nm) | Identifier | Identifier |
| 1 | Fe₃O₄ | 6 | 6 | A | a |
| 2 | Fe₃O₄ | 16 | 16 | B | b |
| 3 | Fe₃O₄ | 24 | 7 | C | c |
| 4 | Fe₃O₄ | 225 | 15 | | d |
| 5 | BaFe₁₂O₁₉ | 10 | 100 | D | e |

### Sample Preparation

Four agar phantoms without contrast agents were prepared using granulated agar (Fisher Brand, molecular genetic granulated): two uniform phantoms with different agar concentrations, and two bilayer phantoms of three percent agar allowed to set in different environments. These phantoms were prepared to determine the effects of agar gel concentration and the agar gel setting (solidifying process) environment upon the transverse relaxation time (T₂) of agar gels.

The sample preparation conditions are summarized in Table 2. Phantom A-1 was a two percent agar (w/w) phantom made by placing 2 g of agar granules in 100 mL of distilled water in a 250 mL beaker. The solution was stirred at 1000 rpm and heated to 90° C. until a thick clear viscous solution formed. This solution was poured into a disposable culture tube (Fisher Brand, 12 mm×75 mm) and left to solidify at room temperature. The opening was sealed with parafilm after the agar set into a cloudy white gel. Phantom A-2, a three percent gel, was made using the same method. The two bilayer phantoms contained three percent agar in a disposable culture tube. Phantom A-3 was filled half-way with liquid agar that was placed in a freezer to set. A second layer was placed on top of the frozen layer and left to set at room temperature. Parafilm was used to seal the culture tube. Phantom A-4 has a bottom layer of agar allowed to solidify at room temperature and a top layer of agar left on a hot plate for an extended time to evaporate also allowed to set at room temperature. Again, parafilm was used to seal the sample.

TABLE 2
Agar phantoms without contrast made to calculate agar transverse
relaxation time (T₂) as a function of concentration.

| | | | Solidifying |
|---|---|---|---|
| Phantom | Composition | Layer | environment |
| A-1 | 2% agar | | Room temp. |
| A-2 | 3% agar | | Room temp. |
| A-3 | 3% agar | Bottom | Room temp. |
| | 3% agar | Top | Evaporated |
| A-4 | 3% agar | Bottom | Freezer |
| | 3% agar | Top | Room temp. |

Eight contrast agent phantoms were prepared using the four available types of sample material: A, B, C, and D. One set of contrast agent phantoms used a small amount (1% w/w) of sodium dodecyl sulfate (SDS) (purchased from Fluka Chemika) as a particle coating for each type of magnetic material. The other set of phantoms used dioctyl sodium sulfosuccinate (AOT, purchased from Fluka Chemika) to coat the particles. The particular contrast agent phantoms are summarized in Table 3.

TABLE 3
Magnetic material and concentration used in
the phantoms containing contrast agents.

| | | Particle A | Particle B | Particle C | Particle D |
|---|---|---|---|---|---|
| Layer | Coating | [C] (mg/mL) | [C] (mg/mL) | [C] (mg/mL) | [C] (mg/mL) |
| | | Phantom 1 | Phantom 2 | Phantom 3 | Phantom 4 |
| 1 | SDS | 0.00 | 0.00 | 0.00 | 0.00 |
| 2 | SDS | 0.388 | 0.315 | 0.355 | 0.339 |
| 3 | SDS | 0.129 | 0.105 | 0.118 | 0.113 |
| 4 | SDS | 0.0430 | 0.0352 | 0.0394 | 0.0375 |
| 5 | SDS | 0.0144 | 0.0117 | 0.0131 | 0.0125 |
| 6 | SDS | 0.00479 | 0.00391 | 0.00436 | 0.00418 |
| 7 | SDS | 0.00160 | 0.00130 | 0.00146 | 0.00139 |
| 8 | SDS | 0.000534 | 0.000434 | 0.000485 | 0.000464 |
| 9 | SDS | 0.00 | 0.00 | 0.00 | 0.00 |
| | | Phantom 5 | Phantom 6 | Phantom 7 | Phantom 8 |
| 1 | AOT | 0.00 | 0.00 | 0.00 | 0.00 |
| 2 | AOT | 0.315 | 0.318 | 0.357 | 0.309 |
| 3 | AOT | 0.105 | 0.106 | 0.119 | 0.103 |
| 4 | AOT | 0.0350 | 0.0354 | 0.0397 | 0.0343 |
| 5 | AOT | 0.0116 | 0.0118 | 0.0133 | 0.0115 |
| 6 | AOT | 0.00389 | 0.00393 | 0.00442 | 0.00381 |
| 7 | AOT | 0.00130 | 0.00131 | 0.00147 | 0.00126 |
| 8 | AOT | 0.000434 | 0.000436 | 0.000491 | 0.000423 |
| 9 | AOT | 0.00 | 0.00 | 0.00 | 0.00 |

The contrast agent phantom samples were made of 500 μL layers of 3% agar (w/w) with a different concentration (mg/mL) of particles in each layer. FIG. 9 is a schematic of a phantom containing contrast agents. The bottom layer is pure agar upon which the most concentrated layer is placed with each successive layer having a lower concentration of magnetic particles. All agar layers were allowed to set at room temperature before the next layer was added. The remaining volume of each sample tube was filled with pure agar. Parafilm was used to seal the sample tubes.

The multi-layered contrast agent phantoms have nine layers as shown in FIG. 9. The process for constructing a single phantom is given below. First, 100 mL of 3% agar gel was made and kept on a hot plate at 90° C. and agitated with a magnetic stirring rod at 1000 rpm. Second, seven aqueous solutions of magnetic particles were created by initially placing approximately 10 mg of magnetic material in 1 mL of distilled water with approximately 1 mg of SDS or AOT in a 1.5 mL centrifuge tube labeled C1. A sonicator was used to agitate the suspension and coat the particles with the surfactant for approximately 10 minutes. This aqueous concentration was then successively diluted by transferring (pipetting) 500 μL of the prior concentration to a new centrifuge tube, adding 1 mL of distilled water, and then sonicating the suspension. In this way C2-C8 were made. The initial aqueous concentration C1 is not placed in agar and is set aside after this process, the seven dilutions C2-C8 are used in the agar layers. After the aqueous concentrations are made they are placed in a rack and set aside. Third, in a separate rack 9 disposable culture tubes labeled L1 through L9 are placed, the rack is then set in a heat bath at 90° C. 1 mL of agar from the main batch is placed in each tube. To assist with accuracy, a separate sample tube with 1 mL of water was used to create a marker on the agar tubes.

Prior to adding any layers, the phantom sample tube was marked at 500 μL increments. The first layer placed in the phantom was 500 μL of pure agar (L1). It was allowed to fully set before another layer was added to the phantom. The second layer was 500 μL of L2 infused with 100 μL of C2. C2 was sonicated immediately before being placed in the agar of L2. It was then dispersed in the agar by quickly voiding and filling the pipette repeatedly. Afterwards, 500 μL of the suspension were placed in the phantom on top of the first layer of the phantom. The above method was used for each layer containing particles. The top layer was pure agar from L9. Parafilm was used to seal all contrast agent loaded phantoms.

### Imaging and Analysis

The test system for this research was a Bruker Bio spin 7 T, 20 cm horizontal bore magnet MRI machine with a working free space of 8.6 cm for imaging. The software platform provided for the machine was Paravision 4.0 MRI/MRS installed on a personal computer. The entire system is located in a laboratory with a stable room temperature.

MR images were obtained using the software suite Paravision 4.0, which has a predefined multiple slice multiple echo (MSME) imaging sequence; the imaging sequence used on all phantoms to collect data for the transverse relaxations of agar with and without contrast agents. MSME is a CPMG spin echo sequence with the form

90_{χ}-[-TE/2:180_{y}°-TE/2:echo-]ₙ,-TR,

where TE is the echo time, TR is the repetition rate of the sequence, and n is the number of it pulses used to create the echo train. The π/2 and π pulses are given in degrees, and their directional effect (in a rotating reference frame) on the water protons is denoted by the subscripts. The main properties of the sequence are provided in Table 4. The echo time was 10 ms and the repetition time was 2000 ms. There are n=16 rephasing π pulses in the MSME sequence. Signal intensity is monitored at each echo occurring at the intervals of TE.

TABLE 4
Main parameters of the MSME imaging sequence.

| Echo Time | Repetition | | | Slice |
|---|---|---|---|---|
| TE | Time TR | π pulses | Field of | Thickness SI |
| (ms) | (ms) | n | View FOV | (mm) |
| 10 | 2000 | 16 | 8.0/4.0 cm | 1.00 |

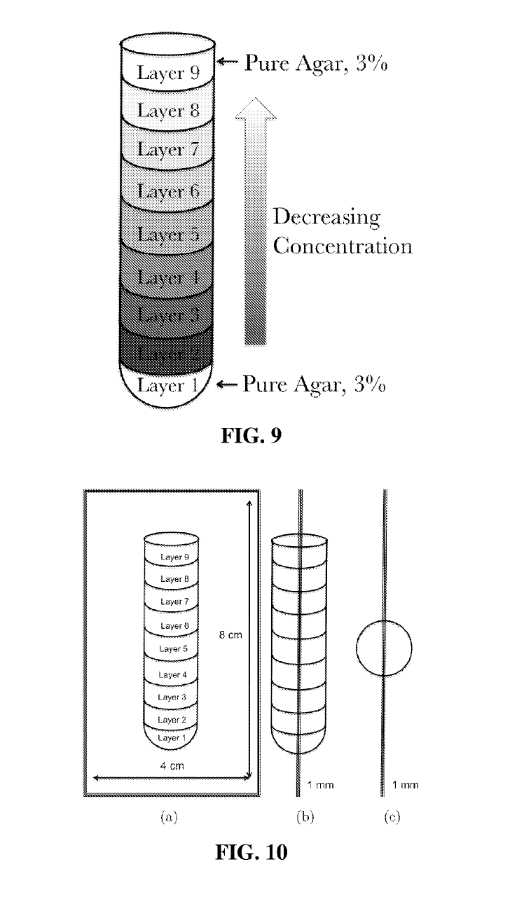

Imaging was limited to a single slice directly through the middle of the phantom sample tube as shown in FIG. 10. The slice was 1 mm thick and was centered on the widest part of the sample tube. The field of view plane is 8.0 cm by 4.0 cm with the sample tube located at the center of the plane. For each sequence, 16 images were formed corresponding to the intervals of TE.

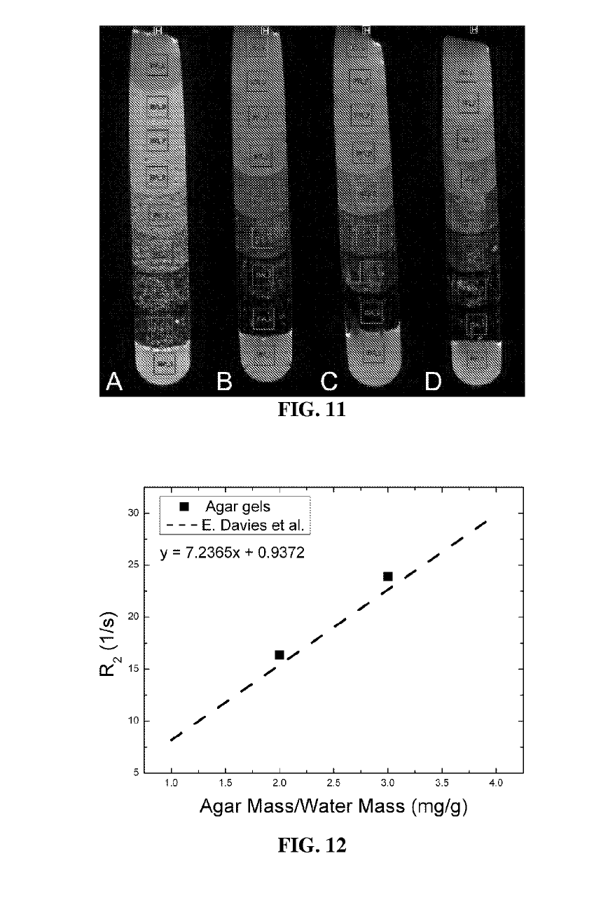

To determine the T₂ times for phantoms containing magnetic particles a square region of interest (ROI) was chosen for each phantom layer. Multiple square ROIs were also used to analyze the agar phantoms not containing contrast agents (FIG. 11) The areas of the ROIs varied from 0.17 cm² to 0.38 cm² and were adjusted to be as large as possible within the layer (similar areas were used for phantoms A-1 through A-4). The signal intensity of the ROIs for each phantom at intervals of the echo time were the collected raw data.

The particle dimensions were confirmed via SEM and are provided in Table 5, along with the density and saturation magnetization MS values known in the art. Images of the particles are provided in FIGS. 4-8. With reference to Table 5, variable “c” refers to one half of the magnetic easy axis length of the particle, and variable “a” refers to one half of the hard axis. The aspect ratio was greater than one for prolate (football shaped) particles, and less than one for oblate (flat disc) particles. The volume (not provided in the table) in all cases was determined using the equation V=4/3πa²c. The effective anisotropy was calculated as the sum of the magnetocrystalline anisotropy and shape anisotropy. The values provided in Table 5 were used for various analyses and calculations disclosed herein.

The measured mean radius of particle 1 was found to be 6 nm, whereas others have indicated that the mean radius should be around 5 nm. The mean radius of particle 2 was well below 50 nm, which is the mean particle radius provided by commercial sources. The dimensions of particles 3 and 4 did not differ substantially from those values known in the art. With reference to particle 5, particular embodiments of this particle were obtained from commercial suppliers. Nevertheless, particular values relating to particle size obtained for these embodiments differed substantially from those values provided by the commercial supplier. For example, in a particular disclosed embodiment, particle 5 length “a” was found to be 100 nm, twice the size as reported by commercial supplier (2a<100 nm). Length “c” for particle 5 was observed as 10 nm. No average thickness for particle 5 were provided by the commercial supplier; however, a radius of an equivalent sphere was provided (R_{ES}=25.4 nm). The observed mean radius of an equivalent sphere was R_{ES}=(a²c)^{(1/3)}=46.5 nm, which is roughly twice the expected value.

TABLE 5
Physical properties of exemplary embodiments.

| | | | | | ρ | M_{S} | K_{eff} |
|---|---|---|---|---|---|---|---|
| | | c | a | | (kg/ | (kA/ | (kJ/ |
| Particle | Composition | (nm) | (nm) | α = c/a | m³) | m) | m³) |
| 1 | Fe₃O₄ | 6 | 6 | 1.00 | 5180 | 446 | 13.5 |
| 2 | Fe₃O₄ | 16 | 16 | 1.00 | 5180 | 446 | 13.5 |
| 3 | Fe₃O₄ | 24 | 7 | 3.43 | 5180 | 446 | 65.6 |
| 4 | Fe₃O₄ | 225 | 15 | 15.0 | 5180 | 446 | 83.5 |
| 5 | BaFe₁₂O₁₉ | 10 | 100 | 0.10 | 5278 | 380 | 258 |

c = one half of the magnetic easy axis length; a = one half of the magnetic hard axis length; α = aspect ratio; ρ = particle density; M_{S} = saturation magnetization of the material; K_{eff} = effective particle anisotropy.

The relaxation times of the pure agar phantoms are given in Table 6. The transverse relaxation times were found by fitting the signal intensity decay for the defined regions of interest (ROIs) of each phantom. The results show that agar is a stable material with a well-defined transverse relaxation time T₂. Agar phantoms A-1 and A-2 were prepared to analyze the transverse relaxation rate of agar as a function of concentration. The relaxation rates R₂ for phantoms A-1 and A-2 were in agreement with other published results as can be seen in FIG. 12. The line shown is that calculated by Davies et al., whose experimental work determined a linear dependence of agar R₂ as a function of agar concentration. The results of A-1 and A-2 match, giving confidence to the R₂ values of the agar gels used in the disclosed phantoms.

Agar phantoms A-3 and A-4 were made to compare the effects of the gel setting under different physical environments. The relaxation times of phantoms A-3 and A-4 were in agreement with that of phantom A-2, T₂=41:8±1:44 ms. The top layer of phantom A-3 (3% agar) was allowed to evaporate on the hot plate for thirty minutes. This amount of time is approximately how long it takes to create an individual agar phantom, and this embodiment was used to determine whether the T₂ of each phantom layer containing contrast would vary due to evaporation. The agar should be more concentrated if some of the water evaporated. A slight decrease in T₂ was observed, but the decrease was not large. The bottom layer of phantom A-4 was placed in a freezer. The freezer was pumping cold air onto the sample as it was allowed to set for ten minutes. Again there was a slight decrease in the T₂ time, but not a significant effect.

TABLE 6
Observed relaxation rate of agar under various conditions.

| | | Solidifying | |
|---|---|---|---|
| Phantom | Composition | Environment | T₂ (ms) |
| A-1 | 2% agar | Room temperature | 61.1 ± 3.43 |
| A-2 | 3% agar | Room temperature | 41.8 ± 1.44 |
| A-3 | 3% agar | Room temperature | 41.4 ± 1.44 |
| | 3% agar | Evaporated | 40.2 ± 1.32 |
| A-4 | 3% agar | Freezer | 40.3 ± 1.17 |
| | 3% agar | Room temperature | 42.3 ± 1.32 |

The observed transverse relaxation times for each phantom layer ROI containing contrast agents are given in Table 7. The results were similar for either surfactant. The results shown hereafter are those for particles coated with SDS. The relaxation times having a linear relationship (e.g., those of lower particle concentrations) were used to create the relaxation rate versus particle concentration plots in FIG. 2 in order to determine the relaxivity r₂ of each type of particle using the equation R₂=1/T₂₀=r₂·C+1/T₂₁. The relaxivities r₂ of the contrast agents are given in Table 8.

These results indicate that, when using the same amount (mass) of these magnetic materials, the oblate barium ferrite spheroids have a greater effect on the observed T₂ than the spherical or prolate magnetite nanoparticles. The transverse relaxivities r₂ of the contrast agents coated with SDS or AOT both rank the contrast mediums used in the order of Particle D, B, C, then A. The barium ferrite oblate ellipsoid (particle D) has the largest effect on the agar transverse relaxivity. The larger of the two spherical particles (particle B) also has a strong effect on the transverse signal intensity, but it is slightly less than that of the barium ferrite platelets. The smaller spheres of magnetite (particle A) provided the lowest relaxivity of the agents studied. The prolate form of magnetite (particle C) has a relaxivity that lies between the smaller and larger sphere effects.

TABLE 7
Transverse relaxation time of samples containing particles A, B, C, and D

| | | Particle A | T₂ | Particle B | T₂ | Particle C | T₂ | Particle D | T₂ |
|---|---|---|---|---|---|---|---|---|---|
| Layer | Coating | (mg/mL) | ms | (mg/mL) | ms | (mg/mL) | ms | (mg/mL) | ms |
| | | Phantom 1 | | Phantom 2 | | Phantom 3 | | Phantom 4 | |
| 1 | SDS | 0.00 | | 0.00 | | 0.00 | | 0.00 | |
| 2 | SDS | 0.388 | 7.7 | 0.315 | 3.445 | 0.355 | 3.805 | 0.339 | 4.174 |
| 3 | SDS | 0.129 | 10.65 | 0.105 | 9.031 | 0.118 | 5.278 | 0.113 | 6.916 |
| 4 | SDS | 0.0430 | 17.16 | 0.0352 | 10.55 | 0.0394 | 11.45 | 0.0375 | 8.712 |
| 5 | SDS | 0.0144 | 23.69 | 0.0117 | 22.01 | 0.0131 | 17.52 | 0.0125 | 18.93 |
| 6 | SDS | 0.00479 | 34.19 | 0.00391 | 38.18 | 0.00436 | 35.85 | 0.00418 | 24.46 |
| 7 | SDS | 0.00160 | 35.16 | 0.00130 | 42.94 | 0.00146 | 38.70 | 0.00139 | 37.29 |
| 8 | SDS | 0.000534 | 32.54 | 0.000434 | 51.90 | 0.000485 | 48.95 | 0.000464 | 48.33 |
| 9 | SDS | 0.00 | | 0.00 | | 0.00 | | 0.00 | |
| | | Phantom 5 | | Phantom 6 | | Phantom 7 | | Phantom 8 | |
| 1 | AOT | 0.00 | | 0.00 | | 0.00 | | 0.00 | |
| 2 | AOT | 0.315 | 6.051 | 0.318 | 4.669 | 0.357 | 3.560 | 0.309 | 4.173 |
| 3 | AOT | 0.105 | 9.208 | 0.106 | 9.062 | 0.119 | 4.161 | 0.103 | 5.667 |
| 4 | AOT | 0.0350 | 20.51 | 0.0354 | 13.49 | 0.0397 | 9.865 | 0.0343 | 8.523 |
| 5 | AOT | 0.0116 | 24.62 | 0.0118 | 21.21 | 0.0133 | 18.75 | 0.0115 | 14.01 |
| 6 | AOT | 0.00389 | 30.95 | 0.00393 | 36.73 | 0.00442 | 26.06 | 0.00381 | 33.27 |
| 7 | AOT | 0.00130 | 32.32 | 0.00131 | 45.70 | 0.00147 | 38.45 | 0.00126 | 41.92 |
| 8 | AOT | 0.000434 | 34.73 | 0.000436 | 52.81 | 0.000491 | 49.70 | 0.000423 | 52.09 |
| 9 | AOT | 0.00 | | 0.00 | | 0.00 | | 0.00 | |

TABLE 8
Transverse relaxivities of disclosed embodiments

| Particle | Axis (major/minor) | r₂ (mL/mg · s) |
|---|---|---|
| A | 6 nm/6 nm | 705.7 ± 80.67 |
| B | 16 nm/16 nm | 2157 ± 46.82 |
| C | 24 nm/7 nm | 1696 ± 226.2 |
| D | 10 nm/100 nm | 2425 ± 153.8 |

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

1. A barium ferrite particle MRI contrast agent having a total hard axis ranging from about 30 nm to about 300 nm and a total easy axis of about 5 nm to about 50 nm.

2. The barium ferrite particle MRI contrast agent according to claim 1 wherein the barium ferrite has a total hard axis ranging from about 30 nm to about 200 nm and a total easy axis of about 5 nm to about 50 nm.

3. The barium ferrite particle MRI contrast agent according to claim 1 wherein the barium ferrite has a total hard axis ranging from about 50 nm to about 150 nm and a total easy axis of about 10 nm to about 40 nm.

4. The barium ferrite particle MRI contrast agent according to claim 1 wherein the barium ferrite has a total hard axis ranging from about 75 nm to about 100 nm and a total easy axis of about 15 nm to about 30 nm.

5. The barium ferrite particle MRI contrast agent according to claim 1 wherein the barium ferrite is a particle having an oblate disc, tube, cone, or combination thereof.

6. The barium ferrite particle MRI contrast agent according to claim 1 wherein the transverse relaxivity ranges from about 2000 mL/mg·s to about 2600 mL/mg·.

7. The barium ferrite particle MRI contrast agent according to claim 1 wherein the transverse relaxivity ranges from about 2100 mL/mg·s to about 2450 mL/mg·s.

8. The barium ferrite particle MRI contrast agent according to claim 1 wherein the barium ferrite comprises one or more pores.

9. A pharmaceutical composition, comprising a pharmaceutically acceptable agent and the barium ferrite particle MRI contrast agent according to claim 1.

10. The pharmaceutical composition according to claim 9 wherein the pharmaceutically acceptable agent is selected from a component that promotes aqueous solubility, a component capable of binding to a target, a component that facilitates biological administration, and combinations thereof.

11. The pharmaceutical composition according to claim 10 wherein the component that promotes aqueous solubility is a polar ligand having a net negative or positive charge or a neutral polar ligand.

12. The pharmaceutical composition according to claim 11 wherein the polar ligand is selected from an optionally substituted ammonium species, an optionally substituted carboxylate species, an optionally substituted alkoxy species, an optionally substituted thio species, an optionally substituted phosphonates, an optionally substituted alkylene oxide, an optionally substituted thiol species, and combinations thereof.

13. The pharmaceutical composition according to claim 10 wherein the component that promotes aqueous solubility is selected from mercaptoacetic acid, mercaptopropionic acid, dihydrolipoic acid, cetyl-trimethylammonium bromide, α-cyclodextrin, polyethylene glycol, cysteamine, cysteine, and combinations thereof.

14. The pharmaceutical composition according to claim 10 wherein the component capable of binding to a target is a specific binding moiety.

15. The pharmaceutical composition according to claim 14 wherein the specific binding moiety is selected from an antibody, a protein, a peptide, a nucleic acid sequence, an enzyme, or combinations thereof.

16. The pharmaceutical composition according to claim 10 wherein the component that facilitates biological administration is a pharmaceutically acceptable excipient selected from surfactants, carbohydrates, lubricants, buffers, osmolality agents, and combinations thereof.

17. The pharmaceutical composition according to claim 9 wherein the pharmaceutically acceptable agent is coupled to the barium ferrite particle MRI contrast agent.

18. The pharmaceutical composition according to claim 17 wherein the pharmaceutically acceptable agent is coupled electrostatically, covalently, or combinations thereof.

19. A method for increasing T₂ contrast in magnetic resonance imaging, comprising:
exposing the barium ferrite particle MRI contrast agent according to claim 1 to a magnetic field.

20. A method for increasing T₂ contrast in magnetic resonance imaging, comprising:
exposing the composition according to claim 9 to a magnetic field.

21. An improved MRI contrast agent comprising a barium ferrite nanoparticle, the improvement being the barium ferrite particle having an oblate shape with a magnetic axis that is perpendicular to a magnetic field, the oblate shape providing the barium ferrite particle with the ability to influence T₂ relaxation without saturation.

22. A method for improving dispersion of the barium ferrite particle MRI contrast agent according to claim 1, comprising modifying the barium ferrite particle MRI contrast agent's magnetic properties, modifying the barium ferrite particle MRI contrast agent's ability to aggregate, or combinations thereof.

23. The method according to claim 22 wherein modifying the barium ferrite particle MRI contrast agent's ability to aggregate comprises encapsulating the metal particle within a polymer colloid, attaching one or more ligands to the barium ferrite particle, exposing the barium ferrite particle to ultra-sonication, high-shear colloid milling, and combinations thereof.

24. The method according to claim 22 wherein modifying the barium ferrite particle MRI contrast agent's magnetic properties comprises changing the barium ferrite particle from a ferromagnetic particle to a superparamagnetic particle by flipping magnetization using temperature control or DC diode sputtering.

25. An improved particle suspension for use in MRI diagnostic procedures, the improvement comprising oblate barium ferrite particles suspended in a medium that prevents nanoparticle aggregation thereby allowing for more accurate imaging by producing varying field gradients capable of magnetically influencing surrounding water molecules and thereby producing an increased signal.

26. A method, comprising:
administering to a subject an effective amount of the barium ferrite particle MRI contrast agent according to claim 1;
exposing the subject, or a tissue or cell of the subject, to a magnetic field; and
detecting a signal produced by the interaction between the barium ferrite particle MRI contrast agent and the magnetic field.

27. The method according to claim 26 wherein administering comprising intravenous or oral administration.

28. The method according to claim 26 wherein detecting comprises obtaining a magnetic resonance image of the subject.

29. The method according to claim 26 wherein the magnetic resonance image is a T₁ weighted image.

30. The method according to claim 26 wherein the magnetic resonance image is a T₂ weighted image.

31. The method according to claim 26 wherein the effective amount ranges from about 0.01 mmol/kg to about 0.1 mmol/kg.

32. The method according to claim 26 wherein the effective amount ranges from about 0.01 mmol/kg to about 0.075 mmol/kg.

33. The method according to claim 26 wherein the effective amount ranges from about 0.01 mmol/kg to about 0.05 mmol/kg.
